(19) United States
(12) Patent Application Publication
(10) Pub. No.: US 2007/0276389 A1
(43) Pub. Date: Nov. 29, 2007

(54) Medical instruments

(76) Inventors: Ralf-Peter Franke, Dornstadt (DE); Michael Fripan, Backnang (DE); Wolfgang Burger, Plochingen (DE); Herbert Richter, Kongen (DE)

Correspondence Address: FULBRIGHT & JAWORSKI, LLP, 666 FIFTH AVE, NEW YORK, NY, 10103-3198, US

(21) Appl. No.: 11/706,097

(22) Filed: Feb. 14, 2007

Related U.S. Application Data
(62) Division of application No. 09/937,722, filed on Mar. 28, 2002, now abandoned.

(30) Foreign Application Priority Data
Apr. 11, 1999 (DE) 199 16 149.6

(51) Int. Cl. A61B 17/56

(52) U.S. Cl. 606/76; 606/1; 606/79; 606/80; 606/82

(57) ABSTRACT

Medical and surgical instruments are made of biocompatible and bioinert materials and used in surgery.

This is a divisional application of U.S. Ser. No. 09/937,722, incorporated herein by reference, which is a §371 of PCT/EP00/03240 filed Apr. 1, 2000 and claims priority from DE 199 16 149.6 filed Apr. 11, 1999.

### BACKGROUND AND SUMMARY OF THE INVENTION

The subject of the present invention is medical instruments, and methods for their manufacture, as well as the use thereof.

Recent studies on patients with implants/prostheses have shown that in the postprosthetic tissue traces of iron could be detected. This finding is surprising insofar as iron could be detected even when implants/prostheses of absolutely iron-free materials have been used and the explanation of the implants/prostheses and the analysis of the periprosthetic tissue was performed with iron-free research instruments. Even in the case of explantates made of absolutely iron-free materials—for example even in the case of titanium prostheses—iron was detected by such studies in the periprosthetic tissue in amounts of up to 1 mg/g of tissue.

The induction effect of iron on fibroblasts, for example, is known. About 30% of the so-called “exchange operations” are made necessary predominantly by particles in the periprosthetic tissue which are responsible for the loosening of implants/prostheses (“particle disease”). Iron, on the one hand an essentially necessary element for the organism, on the other hand exercises evidently massive deleterious effects in the environment of implants/prostheses, e.g., on the ingrowth performance of osteosynthesis plates, implants, prostheses, screws, etc.

Results obtained on the basis of the use—due to the special research methodology—of absolutely iron-free instruments, the iron detected in the periprosthetic tissue of iron-free implants/prostheses must consequently have insinuated themselves during the operation.

Many operational techniques in orthopedics or surgery call for the use of scalpels, scissors, saws, drills, thread cutting tools, centering tools, bushings, templets and other such instruments made of materials containing iron. Consider here, for example, the article, “Semiconstrained Total Elbow Replacement for the Treatment of Post-Traumatic Osteoarthrosis” by A. G. Schneeberger et al., The Journal of Bone and Joint Surgery, Vol. 79-A, No. 8, August 1997, p. 1211 ff.

Surprisingly, in studies of these instruments after their use, definite traces of wear were found. Wear results from the attrition of the ferrous material and sometimes can be seen with the naked eye. This iron-containing detritus created during the operation evidently collects in the periprosthetic tissue and thus can be blamed at least partially for the loosening of the prostheses.

The present invention was therefore aimed at reliably preventing detritus of iron particles from forming in operations.

It was therefore one objective of the invention to make available tools and instruments which, when used in surgical operations, for example in the cutting of bone and in the insertion of implants, will produce no iron particles, in order thus to keep osteolytically active iron out of the tissues.

The problem to which the present invention is addressed was attained according to the invention by the use of biocompatible bioinert materials for the manufacture of medical/surgical instruments and by the use of medical/surgical instruments made from biocompatible bioinert materials in surgical operations.

According to the invention, medical/surgical instruments are prepared from biocompatible bioinert materials.

### BRIEF DESCRIPTION OF THE DRAWINGS p FIG. 12 shows drills prepared according to the invention.

FIGS. 2a and 2b are a comparison between a conventional drill of metal and a drill according to the present invention made of biocompatible bioinert ceramic.

FIG. 3 is a chart showing superior performance of medical/surgical instruments according to the present invention.

The use of biocompatible bioinert materials is of decisive importance for the solution according to the invention. Such biocompatible bioinert materials include ceramics. Examples to mention here are high-strength technical ceramics, such as those on a basis of aluminum oxide, zirconium oxide or silicon nitride. Especially preferred are so-called Y-TZP ceramics or also ZPTA ceramics. ZPTA ceramics consist of a matrix material which is composed of an aluminum oxide/chromium oxide mixed crystal and is platelet-reinforced in situ. Such ceramics are described for example in EPA 0 542 815. These are ceramics in which zirconium dioxide containing stabilizing oxides is embedded in a matrix material of a sintered body formed of an aluminum oxide/chromium dioxide mixed crystal, the amount of the stabilizing oxides being so chosen that the zirconium dioxide is predominantly tetragonal. In addition to these ceramics, however, other ceramics can also be used. It must only be assured that they are biocompatible and bioinert. Such ceramics have long been known in medical technology. They include, among other things, the ceramics from which implants are made, for example, and which are sold by the applicant under the names Biolox® and Ziolox®.

From these high-strength technical ceramics scalpels, scissors, saws, drills, thread cutting tools and centering tools, drill-jig bushings, templets and other such instruments can be made.

The production of the ceramics needed for these instruments is performed in a manner known to the practitioner of the art. It is to be noted, however, that the ceramic required for these instruments must be sharp-edged for use according to the invention in medicine or surgery, and must contain no phase of the kind used in ceramics for cutting metal.

A drill according to the invention is obtained, for example, by first producing a cylinder, from a ceramic according to EPA 0 542 815, for example, into which the shape necessary for use as a cutting instrument is ground. FIG. 1 shows drills which were made in this manner. Likewise possible is the production of a ceramic close to final shape by injection molding methods or by the so-called DCC method, which is then finished accordingly. In the DCC method the green body is made directly from the suspension. For this purpose the ceramic mixture with a solid content of more than 50 vol.-% is ground in an aqueous suspension. The pH value of the mixture is then to be adjusted to 4-4.5. After grinding, urea and a quantity of the enzyme urease is added, which is able to degrade the urea before this suspension is poured into a mold. The enzyme-catalyzed degradation of the urea shifts the pH of the suspension toward 9, while the suspension coagulates. The green body thus prepared is dried and sintered after removal from the mold. The sintering process can be performed without pressure, but pre-sintering followed by hot isostatic compression is also possible. Further details on this process (DCC process) are disclosed in WO 94/02429 and in WO 94/24064, to which reference is expressly made.

A scalpel according to the invention or a scissors according to the invention can be obtained basically according to DE 43 13 305, for example, while the cutting blades of the scissors according to the invention can have either different hardnesses or the same hardness.

According to the invention it is likewise possible to coat known medical/surgical instruments with biocompatible bioinert materials.

In all cases, the appearance, the shape, the geometry, the size of the medical/surgical instruments of the invention can correspond to the medical/surgical instruments used heretofore.

By the use according to the invention of biocompatible bioinert materials for the production of medical/surgical instruments or the use of the medical/surgical instruments consisting of biocompatible bioinert materials in surgical operations it is thus possible for the first time reliably to avoid the entry of iron-containing particles into the tissue. The medical/surgical instruments according to the invention can therefore be used in operations, for example, to avoid the production of any osteolytically active ferrous particles due to the cutting of bone.

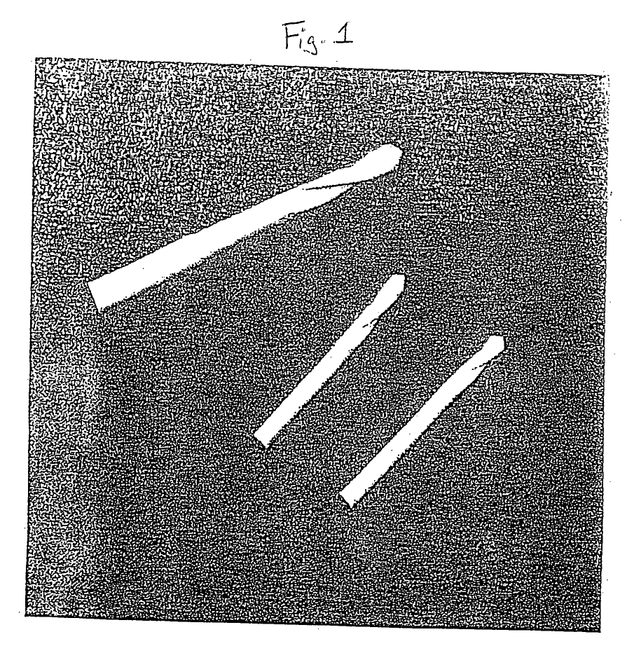

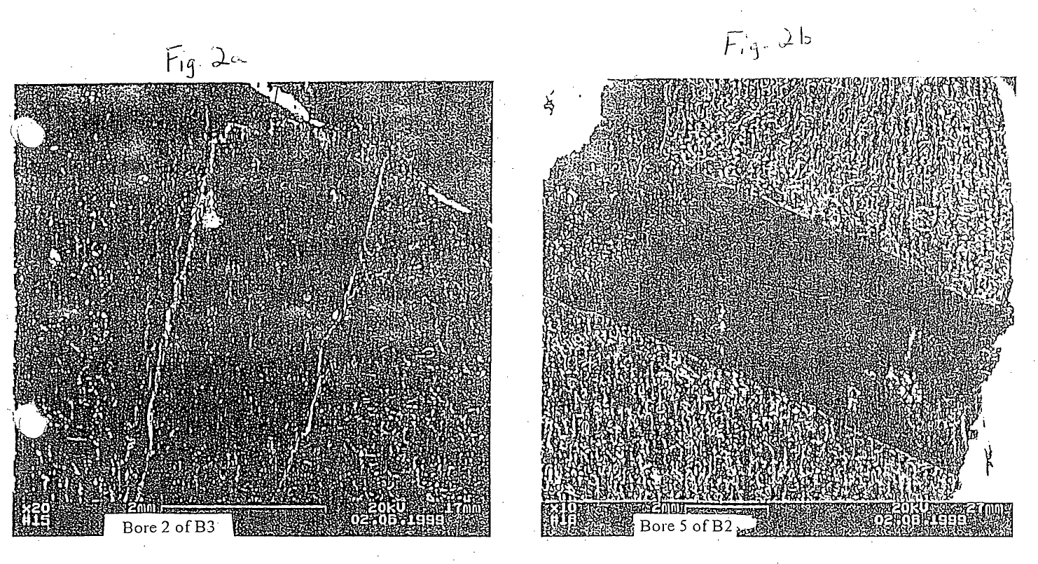

The medical/surgical instruments according to the invention have an extremely great resistance to wear and accordingly high mechanical qualities. It is furthermore advantageous that the cutting characteristic of the medical/surgical instruments according to the invention is substantially better than the cutting characteristic of conventional instruments of the same geometry. FIG. 2 shows the comparison between a conventional drill of metal and a drill according to the invention made of biocompatible bioinert ceramic when used in bone. One reason for this is the surface of the ceramics used according to the invention. Whereas in the case of conventional medical/surgical instruments wettability problems are known to occur when fatty tissue is cut—fatty tissue dulls conventional scalpels, a reason why by now scalpels are used as single-use instruments—this problem does not occur with the medical/surgical instruments according to the invention.

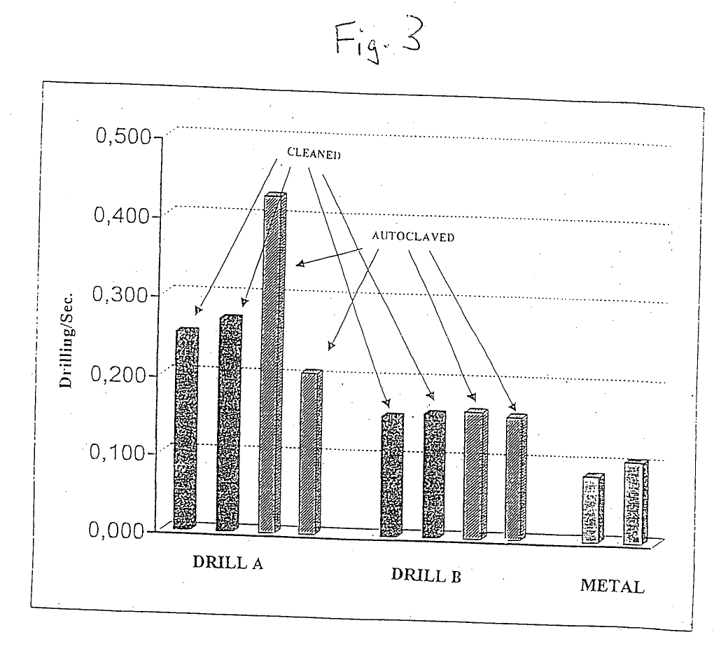

Due to the better cutting characteristic of the medical/surgical instruments of the invention better performance can generally be assumed. Table 1 and FIG. 3 show the comparison of two drills according to the invention with a conventional drill of metal of the same geometry when used in bone.

Of especial, particularly economical importance is furthermore the possibility of being able to use the medical/surgical instruments of the invention more often than once. Conventional instruments of metal can and are, as a rule, used only once. On account of their surface chemistry the medical/surgical instruments of the invention can also be re-sterilized after use, without problems; even if the medical/surgical instruments according to the invention are autoclaved they are superior in performance to the conventional instruments (cf. FIG. 3).

Of especial advantage is furthermore the use of the medical/surgical instruments of the invention in connection with new operation techniques, such as so-called “roboting” or so-called “imaging.” For example, the use of nuclear spin tomography in the operating room makes it necessary to use nonmetallic instruments. Whereas images of metallic instruments are blurred in nuclear spin tomography, the medical/surgical instruments of the invention are imaged with sharp contours.

In connection with this invention, when medical/surgical instruments are mentioned, this is to be understood as including instruments and tools which consist at least in part of biocompatible bioinert materials and are used in medicine/surgery and are intended for the same purpose as the medical/surgical instruments.

TABLE 1

| | | | | Drilling |
|---|---|---|---|---|
| | | Drilling time | Bone thickness | depth/sec |
| Drill | | (sec) | (mm) | (mm/sec) |
| A | cleaned*) | 21 | 5.3 | 0.252B |
| | cleaned*) | 17 | 4.6 | 0.271 |
| | autoclaved | 11 | 4.7 | 0.427 |
| | autoclaved | 33 | 6.8 | 0.206 |
| B | cleaned*) | 37 | 6.7 | 0.154 |
| | cleaned*) | 30 | 6.7 | 0.158 |
| | autoclaved | 40 | 6.5 | 0.163 |
| | autoclaves | 35 | 5.6 | 0.157 |
| Metal | | 90 | 7.0 | 0.084 |
| | | 67 | 7.0 | 0.101 |

*)with protein-dissolving cleaning agent

1-29. (canceled)

30. A method comprising performing a medical procedure with a medical or surgical tool, sterilizing the tool, and reusing the medical or surgical tool in a subsequent procedure, wherein said medical or surgical tool comprises a biocompatible inert material that does not form iron particles during the medical or surgical procedure.

31. A method comprising cutting the bone of a patient with a surgical instrument comprising a biocompatible bioinert material wherein no iron particles are formed during said cutting.

32. The method of claim 31, wherein said surgical instrument is selected from the group consisting of a saw and a drill bit.

33. The method of claim 31, wherein said biocompatible inert material is a ceramic.

34. The method of claim 33, wherein said ceramic is a YTZP ceramic.

35. The method of claim 33, wherein said ceramic is a ZTPA ceramic.

36. The method of claim 31, wherein said bioinert material is provided as a coating on at least a portion of the instrument.

37. The method of claim 31, wherein said bioinert material is silicon nitride.

38. A method comprising performing a surgical operation with a surgical instrument comprising a biocompatible bioinert ceramic material wherein no iron particles are formed during said surgical operations.
